SEQUENCE LISTING (5 sequences)

SEQ ID NO: 1 - protein, 687 aa, Streptomyces sp. X-199-6
ANEMTYEQLARELLLVGPAPTNEDLKLRYLDVLIDNGLNPPGPPKRILIVGAGIAGLVAGDLLTRAGHDVTILEANANRVGGRIKTFHAKKGEPSPFADPAQYAEAGAMRLPSFHPLTLALIDKLGLKRRLFFNVDIDPQTGNQDAPVPPVFYKSFKDGKTWTNGAPSPEFKEPDKRNHTWIRTNREQVRRAQYATDPSSINEGFHLTGCETRLTVSDMVNQALEPVRDYYSVKQDDGTRVNKPFKEWLAGWADVVRDFDGYSMGRFLREYAEFSDEAVEAIGTIENMTSRLHLAFFHSFLGRSDIDPRATYWEIEGGSRMLPETLAKDLRDQIVMGQRMVRLEYYDPGRDGHHGELTGPGGPAVAIQTVPEGEPYAATQTWTGDLAIVTIPFSSLRFVKVTPPFSYKKRRAVIETHYDQATKVLLEFSRRWWEFTEADWKRELDAIAPGLYDYYQQWGEDDAEAALALPQSVRNLPTGLLGAHPSVDESRIGEEQVEYYRNSELRGGVRPATNAYGGGSTTDNPNRFMYYPSHPVPGTQGGVVLAAYSWSDDAARWDSFDDAERYGYALENLQSVHGRRIEVFYTGAGQTQSWLRDPYACGEAAVYTPHQMTAFHLDVVRPEGPVYFAGEHVSLKHAWIEGAVETAVRAAIAVNEAPVGDTGVTAAAGRRGAAAATEPMREEALTS

SEQ ID NO: 2 - DNA, 2064 bp, Streptomyces sp. X-119-6
  source 1..2064: mol_type = genomic DNA
gccaacgaaatgacctacgaacagcttgcacgcgaactgttactggtgggaccggcccctaccaacgaggacctcaaactgcgttatctggatgtgctgattgacaatggtctgaacccgccgggcccacccaaacgcattctgatcgtgggtgcaggcatcgcgggtctggtggctggggacttgctgacccgtgcaggccatgatgtaaccatccttgaggcgaacgcgaatcgcgtgggtgggcggatcaaaacctttcatgcgaagaaaggtgaacccagcccgttcgcggacccagcccagtatgccgaagcaggcgctatgcggctgccgtcgtttcacccacttacgctcgccctgatcgataaattgggcttaaaacgtcgcctgttcttcaatgtcgatattgacccgcagaccggaaaccaggatgcgccggttccgccggtcttttataaatcgtttaaagacggtaagacgtggaccaacggtgccccaagccccgaatttaaagagcccgataaacgcaatcatacctggattcgcaccaaccgcgaacaggtacgtcgcgcccaatacgcgactgatccaagtagtattaacgaaggctttcacctgactggctgcgagactcgcttaaccgtatcagacatggtgaatcaagccctggaaccggtgcgcgattattactctgtcaaacaggatgatggcacccgtgtcaacaaaccttttaaggaatggctggccggttgggcggacgtggtccgtgatttcgatggctactctatgggccgcttcttgcgcgagtacgcagaatttagcgatgaagcagtcgaagcaattggcacgatcgagaatatgacgagtcgcttacatctggccttctttcactcgttcctcgggcgtagcgatatcgacccgcgtgctacatactgggaaatcgagggcggttcacgcatgctgcccgaaactctggctaaggatctgcgtgatcagattgtgatgggtcagcgtatggtacgcttagagtattatgatccaggacgtgacggccatcatggtgaactgactggcccaggtggcccagcggtggccattcaaaccgttccggagggcgaaccgtatgcggcgacccaaacatggaccggggatctcgcgattgtgaccattccgttttcctcactgcgctttgttaaagttacaccgccgttctcctacaagaaacgccgtgcagtaattgaaacgcattacgatcaagccaccaaagttctccttgaattctctcgccgttggtgggaatttaccgaagcggattggaaacgcgaattggacgcgatcgcacctggtctgtacgactactatcagcaatggggcgaagatgatgccgaggccgcgctcgccttaccgcagtcggttcgtaatctgcctacaggtttgctgggcgcacaccctagtgttgatgagtcacgcattggagaggaacaggtagaatactaccgtaattccgaacttcgcggcggagttcgcccggctacgaatgcctatggtggaggttctaccacggacaatccgaaccggtttatgtattatccgagccacccagtgcctggtacgcaaggcggggtagtgttggctgcttattcgtggagtgatgatgcggcgcgttgggacagctttgacgacgctgagcgctatgggtatgcgctggaaaacttacaatccgtccatggccgccgcatcgaggttttctacacaggagcaggccagacacagagctggttgcgtgatccgtatgcttgtggggaggcagctgtttatacgcctcatcagatgacggcattccacctggatgtggttcgccccgaaggcccggtgtattttgctggggaacacgttagcctgaaacatgcatggattgagggcgcggtggaaaccgcggtccgggcggcgattgcggtcaacgaagcgccggtgggtgatacgggtgtcactgccgcagctggtcgtcgtggggccgccgcggccactgaaccgatgcgggaagaagcgctgacgtcctaa

SEQ ID NO: 3 - protein, 629 aa, synthetic construct
  REGION 1..629: note = Truncated L-glutamate oxidase mutant
ANEMTYEQLARELLLVGPAPTNEDLKLRYLDVLIDNGLNPPGPPKRILIVGAGIAGLVAGDLLTRAGHDVTILEANANRVGGRIKTFHAKKGEPSPFADPAQYAEAGAMRLPSFHPLTLALIDKLGLKRRLFFNVDIDPQTGNQDAPVPPVFYKSFKDGKTWTNGAPSPEFKEPDKRNHTWIRTNREQVRRAQYATDPSSINEGFHLTGCETRLTVSDMVNQALEPVRDYYSVKQDDGTRVNKPFKEWLAGWADVVRDFDGYSMGRFLREYAEFSDEAVEAIGTIENMTSRLHLAFFHSFLGRSDIDPRATYWEIEGGSRMLPETLAKDLRDQIVMGQRMVRLEYYDPGRDGHHGELTGPGGPAVAIQTVPEGEPYAATQTWTGDLAIVTIPFSSLRFVKVTPPFSYKKRRAVIETHYDQATKVLLEFSRRWWEFTEADWKRELDAIAPGLYDYYQQWGEDDAEAAGGVRPATNAYGGGSTTDNPNRFMYYPSHPVPGTQGGVVLAAYSWSDDAARWDSFDDAERYGYALENLQSVHGRRIEVFYTGAGQTQSWLRDPYACGEAAVYTPHQMTAFHLDVVRPEGPVYFAGEHVSLKHAWIEGAVETAVRAAIAVNEAPVGDTGVTAAAG

SEQ ID NO: 4 - protein, 5 aa, synthetic construct
  REGION 1..5: note = Peptide linker
GGGGS

SEQ ID NO: 5 - protein, 10 aa, synthetic construct
  REGION 1..10: note = Peptide linker
GGGGSGGGGS

(19) United States
(12) Patent Application Publication
(10) Pub. No.: US 2023/0203456 A1
(43) Pub. Date: Jun. 29, 2023

(54) L-GLUTAMATE OXIDASE MUTANT

(71) Applicant: AJINOMOTO CO., INC., Tokyo (JP)

(72) Inventors: Kazutoshi Takahashi, Kanagawa (JP); Hiroki Yamaguchi, Kanagawa (JP); Uno Tagami, Kanagawa (JP)

(73) Assignee: AJINOMOTO CO., INC., Tokyo (JP)

(21) Appl. No.: 17/947,596

(22) Filed: Sep. 19, 2022

Related U.S. Application Data
(63) Continuation of application No. PCT/JP2021/011898, filed on Mar. 23, 2021.

(30) Foreign Application Priority Data
Mar. 24, 2020 (JP) 2020-052809
Aug. 20, 2020 (JP) 2020-139357

(51) Int. Cl. C12N 9/06; C12Q 1/00; C12P 7/50

(52) U.S. Cl. CPC C12N 9/0022; C12Y 104/03011; C12Q 1/005; C12P 7/50

(57) ABSTRACT

The present invention provides an alternative L-glutamate oxidase that allows for measurement of L-glutamate. More specifically, the present invention provides the following L-glutamate oxidase mutant (a) or (b) and the like: (a) an L-glutamate oxidase mutant including an amino acid sequence that has 90% or more identity to an amino acid sequence of SEQ ID NO: 3 and exhibits an activity of oxidizing L-glutamate, except an L-glutamate oxidase including an amino acid sequence of SEQ ID NO: 1; or (b) an L-glutamate oxidase mutant comprising a peptide linker consisting of 1 to 20 amino acid residues which is inserted into one or more sites selected from the group consisting of (1) a site in a region proximity to a boundary between α1 and α2 regions, (2) a site in a region proximity to a boundary between α2 and γ regions and (3) a site in a region proximity to a boundary between γ and β regions in the L-glutamate oxidase mutant (a), and having the activity of oxidizing L-glutamate.

This application is a Continuation of, and claims priority under 35 U.S.C. § 120 to, International Application No. PCT/JP2021/011898, filed Mar. 23, 2021, and claims priority therethrough under 35 U.S.C. § 119 to Japanese Patent Applications Nos. 2020-052809, filed Mar. 24, 2020, and 2020-139357, filed Aug. 20, 2020, the entireties of which, as well as all0 citations cited herein, are incorporated by reference herein. Also, the Sequence Listing filed electronically herewith is hereby incorporated by reference (File name: 2022-09-19T_US-643_Seq_Listing; File size: 15 KB; Date recorded: Sep. 19, 2022).

### BACKGROUND

General Field

The present invention relates to L-glutamate oxidase mutants and the like.

### BRIEF DESCRIPTION OF THE RELATED ART

L-glutamate oxidase (GluOX) is an enzyme that catalyzes the following reaction (EC1.4.3.11):

L-glutamate+02+H₂O→2-oxoglutarate+H₂O₂+NH₃

Previously, GluOX has been known to exhibit an activity as a heteromultimer (α₂β₂γ₂) with different subunits, by analysis based on isolation and purification of protein, but a gene encoding GluOX had not identified. The GluOX gene was then reported to encode an immature protein with a molecular weight of approximately 70 kDa existing as a single αβγ-containing chain, and retaining activity even as a homodimer ((αβγ-containing single chain)₂) with two subunits each containing the αβγ-containing single chain (WO2001/079503). As a result of subsequent detailed analysis, it is now believed that a natural form of GluOX is not only cleaved by a protease into three αβγ segments, but also is cleaved by the protease within the α region to form a unique hetero-octameric 3D structure (α1₂α2₂β₂γ₂) containing two a fragments, referred to as α1 and α2, a β unit, and a γ unit so as to exhibit its activity (Arima J et al., FEBS J. 2009 July; 276(14): 3894-903).

L-glutamate is known as a major umami component in foods and is also an important component present in biological samples such as blood. L-glutamate can be easily measured using GluOX. Therefore, GluOX has been used for measurement of L-glutamate in samples.

### SUMMARY

The purpose of the present invention is to provide an L-glutamate oxidase that enables the measurement of L-glutamate.

A higher activity of oxidizing L-glutamate can be achieved by using a single-chain polypeptide, such as the polypeptide having the amino acid sequence of SEQ ID NO: 3, which lacks both a processing elimination region, such as the region of the 467th to 506th amino acid residues in the amino acid sequence of SEQ ID NO: 1, between the γ and β regions, and a processing elimination region, such as the region of the 670th to 687th amino acid residues in the amino acid sequence of SEQ ID NO: 1, in a region downstream of the β region in a αβγ-containing single-chain GluOX, such as the amino acid sequence of SEQ ID NO: 1, described in Patent Literature 1, relative to an αβγ-containing single-chain GluOX described in WO2001/079503 (FIG. 1 and Examples).

A higher activity of oxidizing L-glutamate can also be achieved by using a single-chain polypeptide with a peptide linker inserted in at least one or more boundary proximity regions, such as a region in proximity to a boundary between α1 and α2 regions, a region in proximity to a boundary between α2 and γ regions, and a region in proximity to a boundary between γ and β regions, in particular, the region in proximity to the boundary between α1 and α2 regions, and the region in proximity to the boundary between α2 and γ regions, in the above single chain polypeptide, relative to the above immature protein (FIG. 1, Example).

Furthermore, several mutants capable of exhibiting higher activities of oxidizing L-glutamate relative to that of the αβγ-containing single-chain GluOX are described herein.

It is an aspect of the present invention to provide an L-glutamate oxidase mutant of (a) or (b): (a) an L-glutamate oxidase mutant of the protein having the amino acid sequence of SEQ ID NO: 1, wherein said mutant comprises an amino acid sequence that has 90% or more identity to the amino acid sequence of SEQ ID NO: 3 and exhibits an activity of oxidizing L-glutamate; or (b) an L-glutamate oxidase mutant able to oxidize L-glutamate, wherein said mutant comprises a peptide linker consisting of 1 to 20 amino acid residues which is inserted into a site selected from the group consisting of: (1) a site in a region in proximity to a boundary between α1 and α2 regions, wherein the site consists of the 349^{th} to 363^{rd} amino acid residue of SEQ ID NO: 3, (2) a site in a region in proximity to a boundary between α2 and γ regions, wherein the site consists of the 372nd to 377th amino acid residues of SEQ ID NO: 3, (3) a site in a region in proximity to a boundary between γ and β regions, wherein the site consists of the 466th to 469th amino acid residues of SEQ ID NO: 3, and (4) combinations thereof.

It is a further aspect of the present invention to provide the L-glutamate oxidase mutant as described above, wherein the L-glutamate oxidase mutant is a mutant of an L-glutamate oxidase derived from a microorganism belonging to the genus Streptomyces.

It is a further aspect of the present invention to provide the L-glutamate oxidase mutant as described above, wherein the microorganism belonging to the genus Streptomyces is Streptomyces sp. X-119-6.

It is a further aspect of the present invention to provide the L-glutamate oxidase mutant as described above, wherein the peptide linker is inserted into either one or both of (1) the site in the region in proximity to the boundary between α1 and α2 regions, or (2) the site in the region in proximity to the boundary between α2 and γ regions.

It is a further aspect of the present invention to provide the L-glutamate oxidase mutant as described above, wherein the site in the region in proximity to the boundary between α1 and α2 regions is a site between 356th and 357th amino acid residues in SEQ ID NO: 3, the site in the region in proximity to the boundary between α2 and γ regions is a site between 376th and 377th amino acid residues in SEQ ID NO: 3, or the site in the region in proximity to the boundary between γ and β regions is a site between 466th and 467th amino acid residues in SEQ ID NO: 3.

It is a further aspect of the present invention to provide the L-glutamate oxidase mutant as described above, comprising a mutation in the protein having the amino acid sequence of SEQ ID NO: 3, wherein said mutation is selected from the group consisting of: A106, C210, Q235, D236, D237, P244, T311, W313, Q333, I334, M336, Q338, R339, T416, A438, K441, Y455, Q456, Q457, L505, P558, C561, P569, and combinations thereof.

It is a further aspect of the present invention to provide the L-glutamate oxidase mutant as described above, comprising a mutation in the protein having the amino acid sequence of SEQ ID NO: 3, wherein said mutation is selected from the group consisting of: A106S, C210S, Q235E, D236E, D237E, P244H, T311S, W313F, Q333E, 1334V, 1334L, M336L, Q338E, R339K, T416S, A438P, K441E, Y455F, Q456R, Q457E, Q457K, L5051, P558A, C561S, P569A, and combinations thereof.

It is a further aspect of the present invention to provide a method of analyzing L-glutamate, the method comprising measuring L-glutamate contained in a test sample using the L-glutamate oxidase mutant as described above.

It is a further aspect of the present invention to provide the method as described above, wherein the L-glutamate is measured using N-ethyl-N-(2-hydroxy-3-sulfopropyl)-3-methoxyaniline (TOOS) and 4-aminoantipyrine and a peroxidase in addition to the L-glutamate oxidase mutant as described above.

It is a further aspect of the present invention to provide a method for producing 2-oxoglutaric acid, the method comprising producing 2-oxoglutaric acid from L-glutamate in presence of the L-glutamate oxidase mutant as described above.

It is a further aspect of the present invention to provide a polynucleotide encoding the L-glutamate oxidase mutant as described above.

It is a further aspect of the present invention to provide an expression vector comprising the polynucleotide as described above.

It is a further aspect of the present invention to provide a transformed microorganism comprising an expression unit containing a polynucleotide encoding the L-glutamate oxidase mutant as described above and a promoter operably linked to the polynucleotide.

It is a further aspect of the present invention to provide the method as described above, comprising producing the L-glutamate oxidase mutant using the transformed microorganism as described above.

It is a further aspect of the present invention to provide a L-glutamate detection reagent or kit comprising the L-glutamate oxidase mutant as described above.

It is a further aspect of the present invention to provide the L-glutamate detection reagent or kit as described above, further comprising one selected from the group consisting of a buffer solution or buffer salt for reaction, a hydrogen peroxide detection reagent, an ammonia detection reagent, a 2-oxoglutaric acid detection reagent, and combinations thereof.

It is a further aspect of the present invention to provide a detection system for analysis of L-glutamate, the detection system comprising: (a) a device; and (b) the L-glutamate oxidase mutant according to any of to.

It is a further aspect of the present invention to provide the detection system for analysis of L-glutamate as described above, the system further comprising (c) one selected from the group consisting of a buffer solution or buffer salt for reaction, a hydrogen peroxide detection reagent, an ammonia detection reagent, a 2-oxoglutaric acid detection reagent, and combinations thereof.

It is a further aspect of the present invention to provide an enzyme sensor for analysis of L-glutamate, the enzyme sensor comprising: (a) an electrode for detection; and (b) the L-glutamate oxidase mutant as described above that is immobilized or retained on the electrode for detection.

The L-glutamate oxidase mutant as described herein has a superior activity against L-glutamate and is therefore useful for fast and sensitive measurement of L-glutamate and/or production of 2-oxoglutarate. The L-glutamate oxidase mutant as described herein is easily prepared because no protease treatment is required and no subsequent purification process is required for the protease treated product. An analytical method as described herein is useful in a wide range of fields such as biological research, health nutrition, medication, and food production.

### BRIEF DESCRIPTION OF DRAWINGS

FIG. 1 represents the relationship between processing of L-glutamate oxidase (GluOX) and L-glutamate oxidase mutant. The GluOX gene naturally expresses a single-chain polypeptide that encodes a regions (al region and α2 region), p region and γ region. In nature, this single-chain polypeptide is known to be cleaved by processing with a protease expressed by a microorganism that naturally produces this single-chain polypeptide, such as a microorganism belonging to the genus Streptomyces, to form a hetero-octamer: α1₂α2₂β₂γ₂. The GluOX mutant refers to (A) a truncated single-chain polypeptide lacking both a processing elimination region between γ and β regions and a processing elimination region downstream of the β region in the single-chain polypeptide, and (B) a single-chain polypeptide with a peptide linker inserted in one or more of (1) a site in a region in proximity to a boundary between α1 and α2 regions, (2) a site in a region in proximity to a boundary between α2 and γ regions, and (3) a site in a region in proximity to a boundary between γ and β regions in the truncated single-chain polypeptide (A).

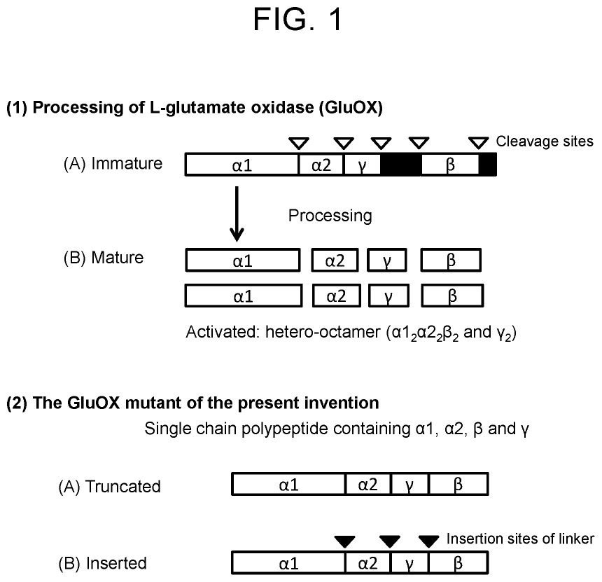

FIG. 2 represents the amino acid sequence of the wild-type GluOX of Streptomyces sp. X-119-6 (SEQ ID NO: 1). The amino acid sequence of this wild-type GluOX (SEQ ID NO: 1) is encoded by the nucleotide sequence of SEQ ID NO: 2. Each of regions in the amino acid sequence of SEQ ID NO: 1 corresponds to the following α-region: a region of the 1st to 376th amino acid residues, wherein the α1 region is the 1st to 352nd amino acid residues; the α2 region is the 361st to 376th amino acid residues; γ-region: a region of the 377th to 466th amino acid residues; and β-region: a region of the 507th to 669th amino acid residues. Protease cleavage sites (indicated by black triangles) in the amino acid sequence of SEQ ID NO: 1 correspond to the following: Protease cleavage region in a region: a region between 353rd to 360th amino acid residues (underlined); a boundary site between α and γ regions: a site between 376th and 377th amino acid residues; a downstream site of γ region: a site between 466th and 467th amino acid residues; an upstream site of 3 region between 506th and 507th amino acid residues; a downstream region of β region: a region between 664th and 669th amino acid residues (underlined). Preferred positions at which point mutation can be introduced for the L-glutamate oxidase mutants are A106, C210, Q235, D236, D237, P244, T311, W313, Q333, 1334, M336, Q338, R339, T416, A438, K441, Y455, Q456, Q457, L545, L598, C601 and P609.

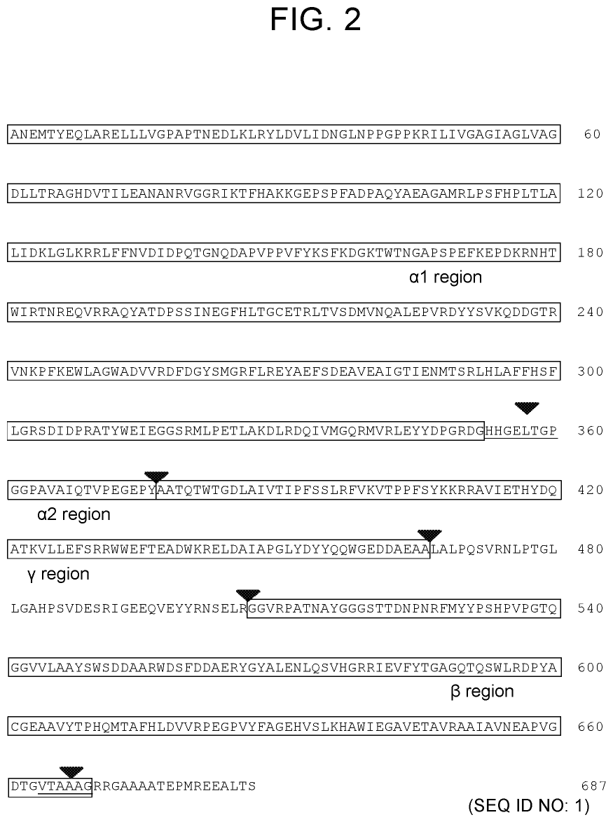

FIG. 3 represents a truncated single-chain polypeptide lacking both a processing elimination region between the γ and β regions, such as the region of the 467th to 506th amino acid residues in the amino acid sequence of SEQ ID NO: 1, and a processing elimination region downstream of the β region, such as the region of the 670th to 687th amino acid residues in the amino acid sequence of SEQ ID NO: 1, in the amino acid sequence of the wild-type GluOX of Streptomyces sp. X-119-6 (SEQ ID NO: 1) (polypeptide having the amino acid sequence of SEQ ID NO: 3). Each of regions in the amino acid sequence of SEQ ID NO: 3 corresponds to the following. α-region: a region of the 1st to 376th amino acid residues, such as the α1 region which is the 1st to 352nd amino acid residues; the α2 region which is the 361st to 376th amino acid residues; the γ-region which is the 377th to 466th amino acid residues; and the β-region which is the 467th to 629th amino acid residues. Each of proximity regions in the amino acid sequence of SEQ ID NO: 3 corresponds to the following: A region in proximity to a boundary between α1 and α2 regions: a region of the 349th to 363rd amino acid residues; a region in proximity to a boundary between α2 and γ regions: a region of the 372nd to 377th amino acid residues; and a region in proximity to a boundary between γ and β regions: a region of the 466th to 469th amino acid residues. The underlined areas correspond to the protease cleavage regions within the α region described in FIG. 2; and the downstream region of the β region. Exemplary positions at which a point mutation can be introduced for the L-glutamate oxidase mutants are A106, C210, Q235, D236, D237, P244, T311, W313, Q333, 1334, M336, Q338, R339, T416, A438, K441, Y455, Q456, Q457, L505 (corresponding to L545 in SEQ ID NO: 1), P558 (corresponding to L598 in SEQ ID NO: 1), C561 (corresponding to C601 in SEQ ID NO: 1), and P569 (corresponding to P609 in SEQ ID NO: 1).

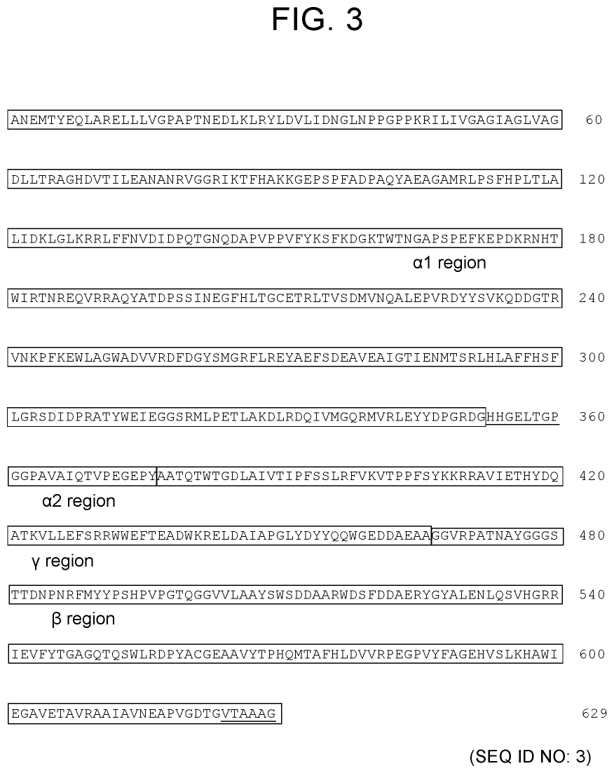

### DETAILED DESCRIPTION OF THE EXEMPLARY EMBODIMENTS

The present invention provides an L-glutamate oxidase mutant of (a) or (b):

(a) an L-glutamate oxidase mutant that includes an amino acid sequence that has 90% or more identity to the amino acid sequence of SEQ ID NO: 3 and exhibits an activity of oxidizing L-glutamate, except an L-glutamate oxidase having the amino acid sequence of SEQ ID NO: 1; or (b) an L-glutamate oxidase mutant that includes a peptide linker of 1 to 20 amino acid residues which is inserted into (1) a site in a region in proximity to a boundary between α1 and α2 regions, (2) a site in a region in proximity to a boundary between α2 and γ regions, and (3) a site in a region in proximity to a boundary between γ and β regions in the L-glutamate oxidase mutant (a), and having the activity of oxidizing L-glutamate, wherein the site in the region in proximity to the boundary between α1 and α2 regions is a site in a region of the 349th to 363rd amino acid residues of SEQ ID NO: 3, the site in the region in proximity to the boundary between α2 and γ regions is a site in a region of the 372nd to 377th amino acid residues of SEQ ID NO: 3, and the site in the region in proximity to the boundary between γ and β regions is a site in a region of the 466th to 469th amino acid residues of SEQ ID NO: 3.

The L-glutamate oxidase mutant (a) (polypeptide of the amino acid sequence of SEQ ID NO: 3) corresponds to a truncated single chain polypeptide lacking both a processing elimination region between γ and β regions, such as a region the 467th to 506th amino acid residues in the amino acid sequence of SEQ ID NO: 1, and a processing elimination region downstream of the β region, such as a region of the 670th to 687th amino acid residues in the amino acid sequence of SEQ ID NO: 1, in the single-chain polypeptide naturally expressed by a GluOX gene (the amino acid sequence of SEQ ID NO: 1). Therefore, the L-glutamate oxidase mutant (a) does not contain the regions of the 467th to 506th and the 670th to 687th amino acid residues in the amino acid sequence of SEQ ID NO: 1.

A percentage of the amino acid sequence identity to the amino acid sequence of SEQ ID NO: 3 in the L-glutamate oxidase mutant (a) may be 92% or more, 95% or more, 97% or more, 98%, or 99% or more. The percentage of the identity of the amino acid sequence can be calculated with GENETYX Ver. 13.1.1 software available from GENETYX CORPORATION by performing Muscle alignment, ClustalW alignment or Multiple sequence alignment using a full length of a polypeptide part encoded in ORF and then calculating while gaps are taken into account to obtain a value for use.

In the L-glutamate oxidase mutant (b), the region in which the peptide linker is inserted refers to one or more (e.g., one, two or three) of the following sites: (1) the site in the region in proximity to the boundary between the α1 and α2 regions, such as the 349th to 363rd amino acid residues in SEQ ID NO: 3, (2) the site in the region in proximity to the boundary between the α2 and γ regions, such as the 372nd to 377th amino acid residues in SEQ ID NO: 3, and (3) the site in the region in proximity to the boundary between the γ and β regions, such as the 466th to 469th amino acid residues in SEQ ID NO: 3. As a particular example, the region in which the peptide linker is inserted may be either one or both of (1) the site in the region in proximity to the boundary between the α1 and α2 regions, and (2) the site in the region in proximity to the boundary between the α2 and γ regions.

The site in the region in proximity to the boundary between the α1 and α2 regions can be the site in the boundary region between the α1 and α2 regions, such as the 349th to 363rd amino acid residues in SEQ ID NO: 3, or the site between the 356th to 357th amino acid residues in SEQ ID NO: 3.

The site in the region in proximity to the boundary between the α2 and γ regions is the site in the region in proximity to the boundary between the α2 and γ regions, such as the 372nd to 377th amino acid residues in SEQ ID NO: 3, or the site between the 376th to 377th amino acid residues in SEQ ID NO: 3.

The site in the region in proximity to the boundary between the γ and β regions is the site in the region in proximity to the boundary between the γ and β regions, such as of the 466th to 469th amino acid residues in SEQ ID NO: 3, or the site between the 466th and 467th amino acid residues in SEQ ID NO: 3.

A peptide linker is a peptide linker of 1 to 20 amino acid residues. The peptide linker may have an amino acid sequence that is different from a partial amino acid sequence of the L-glutamate oxidase. The peptide linker may have two or more, three or more, four or more, or five or more amino acid residues. The peptide linker may have 18 or fewer, 16 or fewer, 14 or fewer, 12 or fewer, or 10 or fewer amino acid residues. More specifically, the peptide linker may have 2 to 18, 3 to 16, 4 to 14, 5 to 12, or 5 to 10 amino acid residues.

The types of amino acid residues constituting the peptide linker can be glycine (G) residues and residues of the naturally-occurring L-α-amino acids that make up general proteins. Specific examples of the residues of such a naturally-occurring L-α-amino acid include L-alanine (A), L-asparagine (N), L-cysteine (C), L-glutamine (Q), L-isoleucine (I), L-leucine (L), L-methionine (M), L-phenylalanine (F), L-proline (P), L-serine (S), L-threonine (T), L-tryptophan (W), L-tyrosine (Y), L-valine (V), L-aspartic acid (D), L-glutamic acid (E), L-arginine (R), L-histidine (H) and L-lysine (K). The type of amino acid residue constituting the peptide linker can be amino acid residues capable of easily constituting a peptide linker with high flexibility. Examples of the amino acid residues capable of easily constituting the peptide linker with high flexibility include glycine (G), L-alanine (A), L-serine (S), and L-threonine (T).

The peptide linker can be GGGGS (SEQ ID NO: 4) or a repeated sequence thereof. The number of repetitions in the repeated sequence can be two to four, two or three, or two.

The activity of oxidizing L-glutamate is an activity that catalyzes the following reaction.

L-glutamate+O₂+H₂O→2-oxoglutarate+H₂O₂+NH₃

The L-glutamate-oxidizing activity of the L-glutamate oxidase mutant as described herein is not particularly limited as long as it is comparable to or higher than that of the L-glutamate oxidase having the amino acid sequence of SEQ ID NO: 1, but can be 1.1-fold or higher L-glutamate-oxidizing activity, or 1.2-fold or higher, as compared to that of the L-glutamate oxidase having the amino acid sequence of SEQ ID NO: 1. Such an activity of oxidizing L-glutamate can be measured by utilizing the oxidation reaction of L-glutamate (10 mM) and its conjugation reaction, as described in Examples.

Oxidation reaction of L-glutamate: A reaction catalyzed by L-glutamate oxidase:

L-glutamate+O₂+H₂O→2-oxoglutarate+H₂O₂+NH₃

Conjugation reaction: Reaction catalyzed by peroxidase
H₂O₂+TOOS+4-AA→dye compound (absorbance: approx. 555 nm)
TOOS: N-ethyl-N-(2-hydroxy-3-sulfopropyl)-3-methoxyaniline

### 4-AA: 4-Aminoantipyrine

The L-glutamate-oxidizing activity of the L-glutamate oxidase mutants as described herein is not particularly limited as long as they exhibit a higher L-glutamate-oxidizing activity than an αβγ-containing single-chain L-glutamate oxidase described in WO2001/079503. The L-glutamate-oxidizing activity of the L-glutamate oxidase mutant may be comparable to or higher than that of the L-glutamate oxidase having the amino acid sequence of SEQ ID NO: 3. The L-glutamate-oxidizing activity of the L-glutamate oxidase mutant may be 1.1-fold or more, 1.2-fold or more, 1.3-fold or more, 1.4-fold or more, 1.5-fold or more, 1.6-fold or more, 1.7-fold or more, or 1.8-fold or more higher than that of an αβγ-containing single-chain L-glutamate oxidase described in WO2001/079503 or the L-glutamate oxidase having the amino acid sequence of SEQ ID NO: 3. Such an oxidizing activity of L-glutamate can be measured by utilizing the oxidation reaction of L-glutamate (10 mM) and its conjugation reaction, as described above.

The L-glutamate oxidase mutant may have one or more mutations capable of enhancing the oxidizing activity of the L-glutamate oxidase mutant. Examples of such mutations include one or more of the following mutations: A106, C210, Q235, D236, D237, P244, T311, W313, Q333, 1334, M336, Q338, R339, T416, A438, K441, Y455, Q456, Q457, L505, P558, C561 and P569 in SEQ ID NO: 3. The amino acid residues obtained after the mutation at these sites are the desired naturally-occurring L-α-amino acid residues that differ from the amino acid residues prior to the mutation. Such a desired naturally-occurring L-alpha-amino acid residue is L-alanine (A), L-asparagine (N), L-cysteine (C), L-glutamine (Q), L-isoleucine (I), L-leucine (L), L-methionine (M), L-phenylalanine (F), L-proline (P), L-serine (S), L-threonine (T), L-tryptophan (W), L-tyrosine (Y), L-valine (V), L-aspartic acid (D), L-glutamic acid (E), L-arginine (R), L-histidine (H), L-lysine (K) or L-glutamic acid (G) residue. When two or more mutations of the L-glutamate oxidase are present, such mutations may include combinations of D236 with another mutation, such as D236 and Q338, D236 and R339, D236 and T416, combinations of T311 with another mutation, such as T311 and D236, T311 and Q457, and combinations of Q457 with another mutation, such as Q457 and D236, Q457 and T311, Q457 and Q338, Q457 and R339.

The one or more mutations capable of enhancing the oxidizing activity of the L-glutamate oxidase mutant can be a mutation or mutations of one or more (e.g., 1, 2, or 3) of the following amino acid residues: A106S, C210S, Q235E, D236E, D237E, P244H, T311S, W313F, Q333E, 1334V, 1334L, M336L, Q338E, R339K, T416S, A438P, K441E, Y455F, Q456R, Q457E, Q457K, L505I, P558A, C561S and P569A in SEQ ID NO: 3. When the L-glutamate oxidase mutants have two or more mutations, such mutations may include combinations of D236E with another mutation, such as D236E and Q338E, D236E and R339K, D236E and T416S, combinations of T311S with another mutation, such as T311S and D236E, T311S and Q457E, and combinations of Q457E with another mutation, such as Q457E and D236E, Q457E and T311S, Q457E and Q338E, Q457E and R339K.

The L-glutamate oxidase mutant as described herein can be derived from any organism, such as microorganisms, animals, and plants, but enzymes from microorganisms belonging to the genus Streptomyces are particular examples, such as Streptomyces sp. X-119-6.

The L-glutamate oxidase mutant may further include another peptide component, such as a tag moiety, at a C-terminus or an N-terminus thereof. Examples of the other peptide component in the L-glutamate oxidase mutant may include peptide components facilitating purification of a targeted protein, such as tag moieties such as histidine tag and strep-tag II, and proteins such as glutathione-S-transferase and maltose-binding protein commonly used for purification of the targeted protein, peptide components, such as Nus-tag, enhancing solubility of the targeted protein, peptide components acting as a chaperon, such as a trigger factor, and peptide components acting as linkers linking the L-glutamate oxidase mutant to either one or both of a protein with another function and a domain thereof.

The modified enzyme can be prepared using a transformed microorganism expressing the L-glutamate oxidase mutant or cell-free system. The transformed microorganism can be prepared by preparing an expression vector and then introducing this expression vector into a host, for example.

The expression vector can include a polynucleotide (e.g., DNA or RNA) encoding the L-glutamate oxidase mutant. In addition to the polynucleotide, the expression vector may further include regions encoding a promoter, terminator, and regions encoding resistant genes against chemicals, such as tetracycline, ampicillin, kanamycin, hygromycin, and phosphinothricin. The expression vector may be a plasmid or an integrative vector. The expression vector may be a viral vector or vectors for cell-free system, as well. The expression vector may further include a polynucleotide(s) which encode(s) another peptide component(s) linked to the L-glutamate oxidase mutant, at the 3′ or 5′ terminus side of the polynucleotide. Examples of the polynucleotide encoding (an)other peptide component(s) include polynucleotides encoding peptide components that facilitate the purification of the target protein as described above, polynucleotides encoding peptide components that improve the solubility of the target protein as described above, polynucleotides encoding peptide components that act as chaperones, and polynucleotides encoding peptide components that act as linkers linking the L-glutamate oxidase mutant to either one or both of the protein with another function and the domain thereof. A variety of expression vectors that contain polynucleotides encoding other peptide components can be used. Therefore, such expression vectors may be used to prepare the expression vectors. Examples of the expression vectors (e.g., pET-15b, pET-51b, pET-41a, and pMAL-p5G) with polynucleotides encoding peptide components that facilitate the purification of the target protein include expression vectors (e.g., pET-50b) with polynucleotides encoding peptide components that improve the solubility of the target protein, expression vectors (e.g., pCold TF) with polynucleotides encoding peptide components that act as chaperones, and expression vectors with polynucleotides encoding peptide components that act as linkers linking the L-glutamate oxidase mutant to either one or both of the protein with another function and the domain thereof. In order to allow for cleavage of the attached (an)other peptide component(s) from the L-glutamate oxidase mutant after the protein expression, the expression vector may include a region encoding a cleavage site to be cleaved by a protease between the polynucleotide encoding the L-glutamate oxidase mutant and the polynucleotide encoding (an)other peptide component(s).

Various prokaryotic cells including cells from bacteria belonging to genera Escherichia (e.g., Escherichia coli), Corynebacterium (e.g., Corynebacterium glutamicum) and Bacillus (e.g., Bacillus subtilis), and various eukaryotic cells including cells from fungi belonging to genera Saccharomyces (e.g., Saccharomyces cerevisiae), Pichia (e.g., Pichia stipitis) and Aspergillus (e.g., Aspergillus oryzae) can be used as the host for expressing the modified enzyme as described herein. In addition, microorganisms, such as microorganisms belonging to the genus Streptomyces, that do not express proteases capable of cleaving L-glutamate oxidase mutants may be suitably used as hosts for expressing the L-glutamate oxidase mutant. A strain in which a certain gene has been deleted may be used as the host. Examples of the transformed microorganism include transformed microorganisms having the vector in its cytoplasm and transformed microorganisms with a targeted gene is integrated into its genome.

The transformed microorganism can be cultured in a medium having a composition described herein using a culture apparatus, such as a test tube, flask, and jar fermenter. Culture conditions can appropriately be determined. Specifically, culture temperature may be 10° C. to 37° C., pH value may be 6.5 to 7.5, and culture period may be 1 to 100 hours. The cultivation may also be carried out while managing the dissolved oxygen concentration. In this case, the dissolved oxygen concentration (DO value) may be used as an indicator for control. A ventilation/stirring condition can be controlled so that a relative dissolved oxygen concentration, a DO value be not below 1 to 10% for example, or not below 3 to 8% when an oxygen concentration in air is 21%. The cultivation may be a batch cultivation or a fed-batch cultivation. In the case of the fed-batch cultivation, the cultivation can also be continued by sequentially adding continuously or discontinuously a solution as a sugar source and a solution containing phosphate to the culture medium.

The host to be transformed is as described above, and for Escherichia coli can be Escherichia coli K12 subspecies Escherichia coli JM109 strain, DH5a strain, HB101 strain, BL21 (DE3) strain, and the like. Methods of performing the transformation and methods of selecting the transformant have also been described in Molecular Cloning: A Laboratory Manual, 3rd edition, Cold Spring Harbor press (2001/01/15), and the like. Hereinafter, a method of making transformed Escherichia coli and producing a predetermined enzyme using this will be described specifically by way of example only.

The promoter for producing a foreign protein in E. coli can generally be one capable of expressing the polynucleotide as described herein. Examples thereof may include potent promoters such as a PhoA, a PhoC, a T7 promoter, a lac promoter, a trp promoter, a trc promoter, a tac promoter, PR and PL promoters of lambda phage, and a T5 promoter; and the PhoA, the PhoC and the lac promoters are particular examples. For example, pUC (e.g., pUC19 or pUC18), pSTV, pBR (e.g., pBR322), pHSG (e.g., pHSG299, pHSG298, pHSG399 or pHSG398), RSF (e.g., RSF1010), pACYC (e.g., pACYC177 or pACYC184), pMW (e.g., pMW119, pMW118, pMW219 or pMW218), pQE (e.g., pQE30) and derivatives thereof may be used as the vector. A vector from phage DNA may also be utilized as the other vector. Furthermore, an expression vector that includes a promoter and can express an inserted DNA sequence may also be used. The vector may be pUC, pSTV, or pMW.

Also, a terminator that is a transcription terminating sequence may be ligated downstream of the polynucleotide. Examples of such a terminator may include a T7 terminator, an fd phage terminator, a T4 terminator, a terminator of a tetracycline resistant gene, and a terminator of Escherichia coli trpA gene.

The vector for introducing the polynucleotide into Escherichia coli can be a so-called multicopy vector, and examples thereof may include plasmids which have a replication origin from ColE1, such as pUC-based plasmids, pBR322-based plasmids and derivatives thereof. Here the “derivative” means those in which the modification has been given to the plasmid by substitution, deletion, insertion and/or addition of base(s).

In order to select the transformed microorganism, the vector can have a marker such as an ampicillin resistant gene. Expression vectors having a potent promoter are commercially available as such a plasmid, such as pUC-based (supplied from Takara Bio Inc.), pPROK-based (supplied from Clontech), or a pKK233-2-based (supplied from Clontech)).

The L-glutamate oxidase mutant can be obtained by transforming Escherichia coli using the resulting expression vector and culturing this Escherichia coli.

Media such as M9/casamino acid medium and LB medium generally used for culturing Escherichia coli may be used as the medium. The medium may contain a predetermined carbon source, nitrogen source, or coenzyme (e.g., pyridoxine hydrochloride). Specifically, peptone, yeast extract, NaCl, glucose, MgSO₄, ammonium sulfate, potassium dihydrogen phosphate, ferric sulfate, manganese sulfate, and the like may be used. The cultivation conditions and production-inducing conditions are appropriately selected depending on the chosen marker, promoter, vector, host, and the like.

The L-glutamate oxidase mutant can be recovered by the following methods. The L-glutamate oxidase mutant can be obtained as a disrupted or lysed product by collecting the transformed microorganism and subsequently disrupting, such as by sonication or homogenization, or lysing, such as by treatment with lysozyme the microbial cells. The modified enzyme can be obtained by subjecting such a disrupted or lysed product to techniques such as extraction, precipitation, filtration, and column chromatography.

A method of analyzing L-glutamate is also described herein. The analysis method can include measuring L-glutamate contained in a test sample using the L-glutamate oxidase mutant.

The test sample is not particularly limited as long as the sample is suspected of containing L-glutamate, and examples thereof may include biological samples, such as blood, urine, saliva, and tears, and/or food and beverage, such as nutrient drinks and amino acid beverages. A concentration of L-glutamate in the test sample may be low, such as concentration less than 1 mM, or 1 μM or more and less than 1 mM, or high, such as a concentration of 1 mM or more, or 1 mM or more and less than 1 M.

The analytical method is not particularly limited as long as L-glutamate can be measured using the L-glutamate oxidase mutant, and may involve detection of the generated 2-oxoglutarate or detection of a side product of NH₃ or H₂O₂ generated together with 2-oxoglutarate. Alternatively, the method may involve detecting a conjugation reaction product that is obtained by conjugation with another reaction. The following conjugation reaction is an example of such a conjugation reaction.

Oxidation reaction of L-glutamate: Reaction catalyzed by L-glutamate oxidase
L-glutamate+O₂+H₂O→2-oxoglutarate+H₂O₂+NH₃
Conjugation reaction: Reaction catalyzed by peroxidase
H₂O₂+TOOS+4-AA→dye compound (absorbance: approx. 555 nm)
TOOS and 4-AA are the same as above.

In the case of utilizing the above conjugation reaction, L-glutamate can be measured using TOOS, 4-AA and peroxidase, in addition to the L-glutamate oxidase mutant. Specifically, the test sample is mixed with TOOS, 4-AA and peroxidase in an aqueous solution (e.g., buffer). The mixed sample is then subjected to the enzymatic reaction described above. Finally, the absorbance of the produced (about 555 nm) is detected from the resulting dye compound for the measurement of L-glutamate. The measurement can be performed qualitatively or quantitatively. The measurement may be performed according to an endpoint method in which the measurement is performed until all substrates are reacted, or a rate method (initial rate method), for example. Because of a small amount of oxygen required in the oxidation reaction, with an adequate amount of oxygen dissolved in the reaction system, there is generally no need to compulsorily supply oxygen or oxygen-containing gases into the reaction system.

The L-glutamate oxidase mutant is not or poorly reacts with amino acids (L-α-amino acid) other than L-glutamate. Therefore, even when not only L-glutamate but also other amino acids are present in the test sample, an amount of L-glutamate in the test sample can be specifically evaluated by using the L-glutamate oxidase mutant.

The use of a hydrogen peroxide electrode with the L-glutamate oxidase mutant enables the specific evaluation of the amount of L-glutamate in the test sample.

Furthermore, a kit is described herein that is used for the analysis of L-glutamate and includes (A) the L-glutamate oxidase mutant as described herein.

The kit can further include at least one of (B) a buffer solution or a buffer salt for reaction, (C) a hydrogen peroxide detection reagent, (D) an ammonia detection reagent, and (E) a 2-oxoglutarate detection reagent.

The buffer solution or the buffer salt for the reaction is used for keeping a pH value in a reaction solution suitable for an objective enzymatic reaction.

(C): The hydrogen peroxide detection reagent is used when hydrogen peroxide is detected by coloration or fluorescence, for example. Examples of a combination of the peroxidase with a chromogenic agent capable of serving as a substrate for the peroxidase specifically include a combination of horseradish peroxidase with 4-aminoantipyrine and phenol, or the like, but the use of the hydrogen peroxide detection reagents is not limited to these combinations.

(D): The ammonia detection reagent can be used in an indophenol method with combination use of phenol and hypochlorite, for example.

(E): An example of 2-Oxoglutarate detection reagent is 2-oxoacid reductase.

A detection system is described herein that is used for the analysis of L-glutamate and includes (a) a device and (b) the L-glutamate oxidase mutant.

The L-glutamate oxidase mutant may be present as a unit independent of a microdevice suppliable for use in the device, and may be injected, fixed or placed into the device beforehand. The L-glutamate oxidase mutant is provided while injected, fixed, or placed into the device beforehand. The L-glutamate oxidase mutant is fixed or placed into the device in a direct or indirect way. For example, a microdevice such as a microfluidic chip equipped with a flow channel can be used as the device.

The detection system for the analysis of L-glutamate may further include one or more of (c) the buffer solution or the buffer salt for reaction, the hydrogen peroxide detection reagent, the ammonia detection reagent and the 2-oxoglutarate detection reagent. The detection system for the analysis of L-glutamate may be provided while all of the components (c) are accommodated in the device. Alternatively, it can be provided by accommodating some of the components (c) in the device while not accommodating the remaining component in the device, such as by accommodating it in a different container. In this case, the component (c) not accommodated in the device may be injected for use into the device during measurement of a target substance.

Examples of the device include: 1) a device, such as a device used for steps of mixing and detecting in different sections, provided with a first section allowing the sample to be mixed with the component (c) for preparation of a mixture solution and a second section allowing the prepared mixture solution to come into contact with the L-glutamate oxidase mutant for the purpose of the detection of L-glutamate; 2) a device, such as a device used for the steps of mixing and detecting in a common section, provided with a section allowing the sample to be mixed with the component (c) and the L-glutamate oxidase mutant for the purpose of the detection of L-glutamate with use of the L-glutamate oxidase mutant; and 3) a device, such as one configured to automatically mix the sample and the like that are flowed through a flow channel by sample injection via an inlet of the device, and automatically detect L-glutamate contained in the resulting mixture solution in a detection section, provided with the flow channel allowing the sample to be mixed with the component (c) and the L-glutamate oxidase mutant, as required and a section allowing L-glutamate to be detected with use of the L-glutamate oxidase mutant. From the viewpoint of automation, the device 3), particularly the device 3) with a microfluidic device, is preferred. In the device (3), the L-glutamate oxidase mutant may be provided in the solution flowing through the flow channel or provided while immobilized or retained in the detection section, but can be provided while immobilized or retained in the detection section.

An enzyme sensor that is used for the analysis of L-glutamate and includes (a) an electrode for detection and (b) the L-glutamate oxidase mutant of the present invention immobilized or retained on the detection electrode is also described herein. The L-glutamate oxidase mutant is immobilized or retained on the electrode directly or indirectly.

For example, a hydrogen peroxide detection electrode can be used as the above detection electrode. More specific examples of the detection electrode include an enzymatic hydrogen peroxide detection electrode and a diaphragm hydrogen peroxide detection electrode. This case allows hydrogen peroxide to be detected after produced as a result of the oxidation of L-glutamate by the L-glutamate oxidizing activity, thereby enabling the analysis of L-glutamate. Other components can be used as they are in a configuration employed in well-known sensors, or modified as appropriate for use.

### EXAMPLES

The present invention is described below in detail with reference to Examples, but the present invention is not limited to the following Examples.

Preparation of GluOX Mutant

Each GluOX mutant was prepared as follows. First, the gene for GluOX (SEQ ID NO: 3) was chemically synthesized and cloned into a NdeI-HindIII cloning site of pET-16b (Merck & Co., Inc.). Hereinafter, a plasmid containing the GluOX sequence (SEQ ID NO: 3) with His-tag added to its N-terminus is referred to as pET-16b-GluOX. A reference GluOX (SEQ ID NO: 1) was also prepared in the same way. GluOX mutant was prepared using the KAPA HiFi HotStart ReadyMix PCR kit (NIPPON Genetics Co, Ltd.). The mutation was introduced into the GluOX gene using pET-16b-GluOX as a template according to a general protocol of site-directed mutagenesis. For all cases of single mutations of specific amino acids, insertions of specific amino acid residues and deletions of specific amino acid residues, mutated plasmids were prepared according to this Method.

Expression and purification of GluOX

A recombinant expression system for the reference GluOX and GluOX mutant was constructed using E. coli, and all were prepared by the same method. Herein, a method for expression and purification of GluOX of SEQ ID NO: 3 is described. Transformants of pET-16b-GluOX were obtained from E. coli BL21 (DE3) according to a standard method. Hereinafter, the transformant of BL21 (DE3) by pET-16b-GluOX is referred to as pET-16b-GluOX-BL21 (DE3).

The GluOX was prepared as follows. First, pET-16b-GluOX-BL21 (DE3) is transferred from a glycerol stock thereof onto an LB agar plate containing 100 μg/mL ampicillin for inoculation. Then, the plate was allowed to stand at 37° C. overnight for incubation. Into a 14 mL round tube, 3 mL of LB liquid medium containing 100 μg/mL ampicillin was placed. A single colony in the LB plate was transferred into the medium for inoculation, and then incubated at 37° C. overnight. Into a 50-mL tube, 6 mL of LB liquid medium containing 100 μg/mL ampicillin was placed. Then, 60 μL of culture solution was added to the medium, and incubated at 37° C. by gyratory shaking until the OD₆₀₀ value reached approximately 0.6. The resulting culture solution was allowed to stand at 16° C. for 30 minutes, supplemented with IPTG at a final concentration of 1.0 mM, and then incubated by gyratory shaking at 16° C. overnight. Then, the bacterial cells were collected.

Bacterial cells were suspended in a disruption buffer (50 mM HEPES, 100 mM NaCl, pH 7.5) and then disrupted using an ultrasonic disruptor (BIORUPTOR, Cosmo Bio Co., Ltd.) in an amplitude mode of H for 10 min with 30-second intervals while cooling water was circulated. The sonicated solution was centrifuged at 13,000×g for 15 min at 4° C. After collected, the supernatant was used for purification using a His SpinTrap (GE Healthcare Japan). Buffer used for equilibration and washing was 50 mM HEPES, 100 mM NaCl, pH 7.5. Elution buffer used was 50 mM HEPES, 100 mM NaCl, 500 mM imidazole, pH 7.5. The elution fractions were collected and diluted to 0.01 mg/mL with the elution buffer.

Mutant Screening by Activity Measurement

The activity was evaluated for the reference GluOX and GluOX mutant prepared in Examples 1 and 2 according to the following procedure. First, the absorbance at 555 nm was measured with a microplate reader (Varioskan LUX, Thermo Fisher Scientific, Inc.) for a mixture solution obtained by mixing 100 μL of 0.2 M HEPES, pH 7.5 with 20 μL of 10 mM glutamate solution, 20 μL of 30 mM N-ethyl-N-(2-hydroxy-3-sulfopropyl)-3-methoxyaniline solution (TOOS solution), 2 μL of 0.1 M 4-aminoantipyrine, 2 μL of 1500 U/mL peroxidase and 36 μL of ultrapure water. Then, 20 μL of GluOX solution prepared to 0.01 mg/mL was added, and then the change in the absorbance at 555 nm was measured over time with the microplate reader. Table 1 represents the activities of the reference GluOX and the GluOX mutant (SEQ ID NO: 3) lacking L467 to R506 and R670 to S687, in terms of relative activities with respect to that of the reference GluOX used as a control. Table 2 represents the activities of the reference GluOX and the GluOX mutants formed by inserting or modifying certain amino acid residues in the GluOX mutant (SEQ ID NO: 3) lacking L467 to R506 and R670 to S687, in terms of relative activities with respect to that of the reference GluOX used as a control. Table 3 represents the activities of the GluOX mutants (represented by a residue number in SEQ ID NO: 3) formed by modifying certain amino acid residues in the GluOX mutant (insertion mutant 6) that is formed by inserting GGGGS (SEQ ID NO: 4) between Y376 and A377 and inserting GGGGS (SEQ ID NO: 4) between E356 and L357 in the GluOX mutant lacking L467-R506 and R670-S687 in the GluOX mutant (SEQ ID NO: 3), in terms of relative activities with respect to that of the insertion mutant 6 used as a control.

TABLE 1
Activities of reference GluOX and GluOX mutant
lacking certain amino acid residues

| | | Relative |
|---|---|---|
| ID | GluOX | activity |
| Reference | Control | 100% |
| GluOX | | |
| Deletion | L467 to R506 and R670 to S687 deleted | 123% |
| mutant 1 | in SEQ ID NO: 1 (SEQ ID NO: 3) | |

TABLE 2
Activities of reference GluOX and GluOX mutants (represented by residue
number in SEQ ID NO: 3) formed by inserting and/or modifying specific amino
acid residues in GluOX mutant lacking L467 to R506 and R670 to S687 in
reference GluOX (SEQ ID NO: 3)

| | | Relative |
|---|---|---|
| ID | GluOX | activity |
| Reference | Control | 100% |
| GluOX | | |
| Mutant 1 | W313F | 120% |
| Mutant 2 | A106S | 118% |
| Mutant 3 | L505I | 158% |
| Mutant 4 | C210S | 119% |
| Mutant 5 | C561S | 104% |
| Inserted | GGGGS (SEQ ID NO: 4) inserted between Y376 and | 146% |
| mutant 1 | A377 | |
| Inserted | GGGGS (SEQ ID NO: 4) inserted between E356 and | 146% |
| mutant 2 | L357 | |
| Inserted | GGGGSGGGGS (SEQ ID NO: 5) inserted between E356 | 214% |
| mutant 3 | and L357 | |
| Inserted | GGGGS (SEQ ID NO: 4) inserted between A466 and | 104% |
| mutant 4 | G467 | |
| Inserted | P244H | 213% |
| mutant 6 | | |
| Inserted | GGGGS (SEQ ID NO: 4) inserted between Y376 and | 338% |
| mutant 5 | A377, and P244H mutation | |
| Inserted | GGGGS (SEQ ID NO: 4) inserted between Y376 and | 489% |
| mutant 6 | A377, and GGGGS (SEQ ID NO: 4) inserted between | |
| | E356 and L357 | |
| Inserted | GGGGS (SEQ ID NO: 4) inserted between Y376 and | 150% |
| mutant 7 | A377, and GGGGSGGGGS (SEQ ID NO: 5) inserted | |
| | between A466 and G467 | |
| Inserted | GGGGS (SEQ ID NO: 4) inserted between Y376 and | 316% |
| mutant 8 | A377, D461 to G468 deleted, and GGGGS (SEQ ID NO: | |
| | 4) inserted between E460 and V469 | |
| Inserted | GGGGS (SEQ ID NO: 4) inserted between Y376 and | 266% |
| mutant 9 | A377, and C210S mutation | |
| Inserted | GGGGS (SEQ ID NO: 4) inserted between Y376 and | 225% |
| mutant 10 | A377, GGGGS (SEQ ID NO: 4) inserted between E356 | |
| | and L357, and GGGGSGGGGS (SEQ ID NO: 5) inserted | |
| | between A466 and G467 | |
| Inserted | GGGGS (SEQ ID NO: 4) inserted between Y376 and | 117% |
| mutant 11 | A377, GGGGS (SEQ ID NO: 4) inserted between E356 | |
| | and L357, D461 to G468 deleted, and GGGGS (SEQ ID | |
| | NO: 4) inserted between E460 and V469 | |

TABLE 3
Activities of GluOX mutants (represented by residue number in
SEQ ID NO: 3) formed by modifying certain amino acid residues
in GluOX mutant (insertion mutant 6) that is formed by inserting
GGGGS (SEQ ID NO: 4) between Y376 and A377 and inserting GGGGS
(SEQ ID NO: 4) between E356 and L357 in SEQ ID NO: 3

| | | Relative |
|---|---|---|
| ID | GluOX | activity |
| Inserted | GGGGS (SEQ ID NO: 4) inserted between Y376 | 100% |
| mutant 6 | and A377 and GGGGS (SEQ ID NO: 4) inserted | |
| | between E356 and L357 | |
| Mutant 7 | Q235E | 116% |
| Mutant 8 | D236E | 177% |
| Mutant 9 | D237E | 133% |
| Mutant 10 | Q333E | 157% |
| Mutant 11 | I334V | 158% |
| Mutant 12 | I334L | 187% |
| Mutant 13 | M336L | 111% |
| Mutant 14 | Q338E | 161% |
| Mutant 15 | A438P | 103% |
| Mutant 16 | K441E | 108% |
| Mutant 17 | Y455F | 127% |
| Mutant 18 | Q456R | 120% |
| Mutant 19 | Q457E | 156% |
| Mutant 20 | Q457K | 132% |
| Mutant 21 | P558A | 101% |
| Mutant 22 | P569A | 146% |
| Mutant 23 | P244H | 103% |
| Mutant 24 | D236E and Q338E | 231% |
| Mutant 25 | D236E and R339K | 154% |
| Mutant 26 | D236E and T416S | 290% |
| Mutant 27 | T311S and D236E | 303% |
| Mutant 28 | T311S and Q457E | 184% |
| Mutant 29 | Q457E and D236E | 176% |
| Mutant 30 | Q457E and T311S | 166% |
| Mutant 31 | Q457E and Q338E | 104% |
| Mutant 32 | Q457E and R339K | 133% |

Determination of Boundary Proximity Region

3D structure (PDB ID: 2E1M) of a hetero octamer (α1₂α2₂β₂γ₂) of naturally-occurring GluOX was not observed partially when displayed by PyMOL, a molecular graphics software. It results from uncertain 3D structure that is obtained due to high mobility of the region when experimentally determined by X-ray crystallography for protein 3D structure. Table 4 represents regions of the amino acid residues in SEQ ID NO: 3 where 3D structure was confirmed for the hetero-octamer of naturally-occurring GluOX (α1₂α2₂β₂γ₂). Based on the results, the boundary proximity regions were determined to include the region where the 3D structure was not observed. In other words, the boundary proximity regions in SEQ ID NO: 3 correspond to the regions as follows. A region in proximity to a boundary between α1 and α2 regions: a region consisting of 349th to 363rd amino acid residues; a region in proximity to a boundary between α2 and γ regions: a region consisting of 372nd to 377th amino acid residues; and a region in proximity to a boundary between γ and β regions: a region consisting of 466th to 469th amino acid residues.

TABLE 4
Regions of amino acid residues in SEQ ID NO: 3
where 3D structure was confirmed for hetero-octamer of
naturally-occurring GluOX (α1₂α2₂β₂γ₂)

| Amino acid residues | Whether or not structure of |
|---|---|
| in SEQ ID NO: 3 | amino acid residue is observable |
| Positions 1 to 349 | ∘ |
| Positions 350 to 362 | x |
| Positions 363 to 372 | ∘ |
| Positions 373 to 376 | x |
| Positions 377 to 466 | ∘ |
| Positions 467 to 468 | x |
| Positions 469 to 619 | ∘ |
| Positions 620 to 629 | x |

∘: Yes (observable)
x: No (not observable)

As described above, the L-glutamate oxidase mutant has an improved enzyme activity compared to that of wild type, and is therefore useful for fast and highly sensitive measurement of L-glutamate and/or for production of 2-oxoglutarate and/or for use as a testing reagent for L-glutamate.

### INDUSTRIAL APPLICABILITY

This invention is useful in a wide range of fields, including biological research, health nutrition, medication and food production.

While the invention has been described in detail with reference to exemplary embodiments thereof, it will be apparent to one skilled in the art that various changes can be made, and equivalents employed, without departing from the scope of the invention. Each of the aforementioned documents is incorporated by reference herein in its entirety.

1. An L-glutamate oxidase mutant of (a) or (b):
(a) an L-glutamate oxidase mutant of the protein having the amino acid sequence of SE ID NO: 1, wherein said mutant comprises an amino acid sequence that has 90% or more identity to the amino acid sequence of SEQ ID NO: 3 and exhibits an activity of oxidizing L-glutamate,; or
(b) an L-glutamate oxidase mutant able to oxidize L-glutamate, wherein said mutant comprises a peptide linker consisting of 1 to 20 amino acid residues which is inserted into a site selected from the group consisting of:
(1) a site in a region in proximity to a boundary between α1 and α2 regions, wherein the site consists of the 349^{th} to 363^{rd} amino acid residues of SEQ ID NO: 3,
(2) a site in a region in proximity to a boundary between α2 and γ regions, wherein the site consists of the 372^{nd} to 377^{th} amino acid residues of SEQ ID NO: 3,
(3) a site in a region in proximity to a boundary between γ and β regions, wherein the site consists of the 466^{th} to 469^{th} amino acid amino acid residues of SEQ ID NO: 3, and
(4) combinations thereof.

2. The L-glutamate oxidase mutant according to claim 1, wherein the L-glutamate oxidase mutant is a mutant of an L-glutamate oxidase derived from a microorganism belonging to the genus Streptomyces.

3. The L-glutamate oxidase mutant according to claim 2, wherein the microorganism belonging to the genus Streptomyces is Streptomyces sp. X-119-6.

4. The L-glutamate oxidase mutant according to claim 1, wherein the peptide linker is inserted into either one or both of (1) the site in the region in proximity to the boundary between α1 and α2 regions, or (2) the site in the region in proximity to the boundary between α2 and γ regions.

5. The L-glutamate oxidase mutant according to claim 1,
wherein the site in the region in proximity to the boundary between α1 and α2 regions is a site between 356th and 357th amino acid residues in SEQ ID NO: 3,
the site in the region in proximity to the boundary between α2 and γ regions is a site between 376th and 377th amino acid residues in SEQ ID NO: 3, or
the site in the region in proximity to the boundary between γ and β regions is a site between 466th and 467th amino acid residues in SEQ ID NO: 3.

6. The L-glutamate oxidase mutant according to claim 1, comprising a mutation in the protein having the amino acid sequence of SEQ ID NO: 3, wherein said mutation is selected from the group consisting of A106, C210, Q235, D236, D237, P244, T311, W313, Q333, I334, M336, Q338, R339, T416, A438, K441, Y455, Q456, Q457, L505, P558, C561, P569, and combinations thereof.

7. The L-glutamate oxidase mutant according to claim 1, comprising a mutation in the protein having the amino acid sequence of SEQ ID NO: 3, wherein said mutation is selected from the group consisting of A106S, C210S, Q235E, D236E, D237E, P244H, T311S, W313F, Q333E, 1334V, 1334L, M336L, Q338E, R339K, T416S, A438P, K441E, Y455F, Q456R, Q457E, Q457K, L505I, P558A, C561S, P569A, and combinations thereof.

8. A method of analyzing L-glutamate, the method comprising measuring L-glutamate contained in a test sample using the L-glutamate oxidase mutant according to claim 1.

9. The method according to claim 8, wherein the L-glutamate is measured using N-ethyl-N-(2-hydroxy-3-sulfopropyl)-3-methoxyaniline (TOOS) and 4-aminoantipyrine and a peroxidase in addition to the L-glutamate oxidase mutant according to claim 1.

10. A method for producing 2-oxoglutaric acid, the method comprising producing 2-oxoglutaric acid from L-glutamate in presence of the L-glutamate oxidase mutant according to claim 1.

11. A polynucleotide encoding the L-glutamate oxidase mutant according to claim 1.

12. An expression vector comprising the polynucleotide according to claim 11.

13. A transformed microorganism comprising an expression unit containing a polynucleotide encoding the L-glutamate oxidase mutant according to claim 1 and a promoter operably linked to the polynucleotide.

14. A method of producing the L-glutamate oxidase mutant according to claim 1, comprising producing the L-glutamate oxidase mutant using an expression unit containing a polynucleotide encoding the L-glutamate oxidase mutant and a promoter operably linked to the polynucleotide.

15. An L-glutamate detection reagent or kit comprising the L-glutamate oxidase mutant according to claim 1.

16. The L-glutamate detection reagent or kit according to claim 15, further comprising one selected from the group consisting of a buffer solution or buffer salt for reaction, a hydrogen peroxide detection reagent, an ammonia detection reagent, a 2-oxoglutaric acid detection reagent, and combinations thereof.

17. A detection system for analysis of L-glutamate, the detection system comprising:
(a) a device; and
(b) the L-glutamate oxidase mutant according to claim 1.

18. The detection system for analysis of L-glutamate according to claim 17, the system further comprising (c) one selected from the group consisting of a buffer solution or buffer salt for reaction, a hydrogen peroxide detection reagent, an ammonia detection reagent, a 2-oxoglutaric acid detection reagent, and combinations thereof.

19. An enzyme sensor for analysis of L-glutamate, the enzyme sensor comprising:
(a) an electrode for detection; and
(b) the L-glutamate oxidase mutant according to claim 1 that is immobilized or retained on the electrode for detection.
